(19) United States
(12) Patent Application Publication
(10) Pub. No.: US 2011/0166602 A1
(43) Pub. Date: Jul. 7, 2011

(54) BONE ANCHOR DEVICE

(76) Inventor: Michel H. Malek, Chicago, IL (US)

(21) Appl. No.: 12/882,519

(22) Filed: Sep. 15, 2010

Related U.S. Application Data
(63) Continuation of application No. 11/828,113, filed on Jul. 25, 2007, now abandoned.
(60) Provisional application No. 60/834,011, filed on Jul. 28, 2006.
(60) Provisional application No. 60/841,729, filed on Sep. 1, 2006.

(51) Int. Cl. A61B 17/88; A61B 17/84; A61B 17/86

(52) U.S. Cl. 606/279; 606/300; 606/304

(57) ABSTRACT

Bone anchoring devices for anchoring medical devices to bones and for reducing bone fractures are provided. The devices include a bone anchor portion designed to allow for bony ingrowth and secure fixation to a bone and an internal fastener designed to resist bony ingrowth and facility easy removal of the fastener from a patient.

### CROSS-REFERENCE TO RELATED APPLICATIONS

This application is a continuation of U.S. patent application Ser. No. 11/828,113, filed on Jul. 25, 2007, which claims the benefit of U.S. Provisional Patent Application Nos. 60/834,011, filed Jul. 28, 2006, and 60/841,729, filed Sep. 1, 2006, all of which are hereby incorporated by reference, in their entirety, for any and all purposes.

### FIELD OF THE INVENTION

The invention generally relates to medical devices and anchoring mechanisms. More specifically, a bone anchoring device is disclosed which is capable of anchoring a medical device to bone or reducing a bone fracture with an internal fastener nested within a bone anchor.

### BACKGROUND

In some medical applications, it is required or at least desired, that a medical device be firmly anchored to a bone. For example, in correcting certain conditions of the spine, it is desirable to firmly anchor a spinal stabilization device to one or more vertebra. In such cases, a device may be anchored to a bone via bone screws. The bone screws used in such applications may be constructed of a material which promotes or allows for tissue or bony ingrowth throughout or through a portion of the bone screw, thereby effectively preventing the removal of the bone screw and device from the subject without significant difficulty or trauma to the surrounding bone and tissue. While in some cases it is desired that a medical device permanently remain within a patient, in other cases, after the medical device in a patient has served a useful purpose or needs repair or replacement, it is desirable or necessary to remove the device from a patient's body. Similarly, bone screws may be used in reducing fractures and the like. However, after the repair is complete, removal of the bone screw may or may not be desired.

### SUMMARY

Bone screw devices and anchoring devices for attaching medical devices to bone are provided. In some embodiments, the device comprises a bone anchor comprising an elongated shaft having a cannula running through at least a portion of the length of the shaft. The bone anchor has a proximal end and a distal end, wherein the proximal end may form a head and the distal end may form a tip. In some embodiments, the internal surface of the shaft, which defines the cannula, provides an internal thread. The external surface of the shaft defines an external thread, which allows the bone anchor to be screwed into a bone in order to anchor a device to the bone, or to assist in the healing of a bone fracture. In other embodiments, the internal surface of the shaft comprises at least one retaining member to permanently engage an internal fastener.

Desirably, at least a portion of the external surface of the bone anchor is made from, or coated with, a porous material that allows for bone ingrowth into the material, helping to secure the bone anchor to the bone. In some embodiments, the porous coating is cobalt chromium alloy, cobalt chromium molybdenum alloy, or porous titanium. As an alternative to, or in addition to, the porous material, the bone anchor may include fenestrations, channels or cavities built into its external surface, such that bone growth into the fenestrations, channels or cavities helps to secure the bone anchor to the bone.

The device further includes an internal fastener. In some embodiments the internal fastener is capable of removably being engaged in the cannula of the bone anchor, comprising an elongated shank and having a proximal end, which may form a head, and a distal end, which may form a tip. The elongated shaft of the internal fastener may comprise an external thread on the surface of the elongated shank, thereby defining an internal screw, the proximal end forming a screw head, and the distal end forming a screw tip. In some embodiments, the external thread of the internal screw is designed to compliment the internal thread of the bone anchor such that the internal screw may be screwed into the cannula of the bone anchor. In other embodiments, the internal fastener may be permanently engaged in the bone anchor. In such embodiments, the internal fastener is capable of being permanently engaged in the bone anchor via a mechanism such as lock and key; an annular ring on the elongated shaft internal fastener and at least one retaining member on the internal surface of the cannula of the bone anchor; at least one recess on the elongated shaft of the internal fastener and at least one retaining member on the internal surface of the cannula of the bone anchor; a bayonet style connection; a pin that is inserted through the bone anchor and internal fastener in a direction that is transverse to a longitudinal axis of both the bone anchor and internal fastener; or other mechanism known to those of skill in the art. In some embodiments, the internal fastener is an intramedullary nail.

The internal fastener may be made from a material that does not allow bone ingrowth and, therefore, facilitates the easy removal of the internal fastener (and any devices mounted thereto) from the bone. In some embodiments, the bone anchor is an implant or implantable device, and in other embodiments, the bone anchoring device is an implant or implantable device.

Methods for using the present devices are also provided. In some embodiments, the methods comprise inserting the bone anchor of the device in a bone, engaging the internal fastener in the cannula of the bone anchor, and securing the internal fastener in the bone anchor. In some such embodiments, the engaging comprises screwing the internal screw in the cannula, and the securing comprises tightening the internal screw in the cannula. In some embodiments, the methods further comprise connecting a medical device to a bone with the bone anchoring device. In other embodiments, the methods further comprise removing the internal fastener from the bone anchor, while leaving the bone anchor fixed within a patient's bone. In some embodied methods, where the internal fastener is to be permanently engaged in the bone anchor, the internal fastener is inserted into the cannula of the bone anchor by pushing the annular ring past the retaining member such that the internal fastener is permanently engaged in the bone anchor. In other embodiments, the method comprises pushing the internal fastener into the cannula of the bone anchor until the at least one retaining member engages the at least one recess of the shaft of the internal fastener.

In some embodiments, the combination of a medical device and a bone anchor device comprise a medical device assembly.

### BRIEF DESCRIPTION OF THE DRAWINGS

FIG. 1 is a cross-sectional view of a bone anchor according to one embodiment of the present invention.

FIG. 2 is a side view of an internal fastener according to one embodiment of the present invention.

FIG. 3 is a cross-sectional view of a bone anchor inserted in a bone, an internal screw nested within the bone anchor, and a medical device anchored to the bone according to one embodiment of the present invention.

FIG. 4 is a cross-sectional view of a bone anchor and internal fastener, in which the internal fastener is permanently engaged with the bone anchor, according to one embodiment of the present invention.

FIG. 5 is a cross-sectional view of a bone anchor and internal fastener, in which the internal fastener is permanently engaged with the bone anchor, according to one embodiment of the present invention.

### DETAILED DESCRIPTION

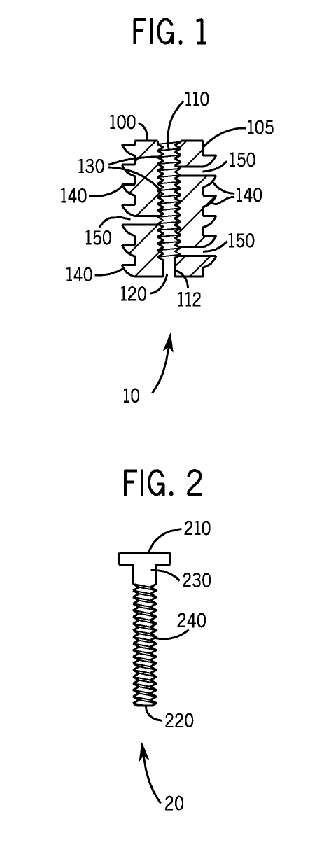

In one aspect of the present invention, a bone anchoring device is provided. The bone anchoring device is a multi-part anchoring device for anchoring orthopedic devices to bone or for use in the reduction of fractures of bones. Referring to FIG. 1, a bone anchoring device comprises at least two parts, a bone anchor 10 and an internal fastener 20. In some embodiments, and as illustrated in FIG. 1, the internal fastener 20 may be a screw, however other fasteners are known to those of skill in the art. For example, other types of fasteners may include, but are not limited to bayonet-style fasteners, lock-and-key mechanism style fasteners, and the like. The bone anchor 10 comprises an elongated hollow shaft 105, the internal surface 112 of which defines a cannula 110. The shaft has a proximal end, which defines the head 100 of the shaft, a distal end, which defines the tip 120 of the shaft, an external thread 140 on the external surface of the elongated hollow shaft 105, and in some embodiments, an internal thread 130 along at least a portion of the internal surface 112. In other embodiments, the internal surface 112 of the shaft comprises a locking mechanism for other types of fasteners described above. The internal fastener 20 comprises an elongated shank 230 having a proximal end, which forms the head 210 of the fastener, a distal end, which forms the tip 220 of the fastener, and, in embodiments where the internal fastener 20 is an internal screw, a thread 240 on at least a portion of the external surface of the elongated shank 230. In some embodiments, the bone anchoring device is used to anchor an orthopedic device to one or more bones.

The bone anchor 10 may comprise a self-tapping tip such that the bone anchor 10 may be started directly into bone without pre-drilling a pilot hole for the bone anchor 10, or a pilot hole may first be drilled. At least a portion of the external surface of the bone anchor (i.e., a portion of the external surface that is to be in contact with a bone) may be coated with, or made from, a porous material that allows for bone ingrowth. As shown in FIG. 1, the bone anchor 10 may also comprise fenestrations 150 that run transverse to the cannula 110. Fenestrations 150 allow for tissue or bone ingrowth into the bone anchor 10. In other embodiments, the bone anchor 10 is an implant or an implantable device.

The head 100 of the bone anchor 10 may comprise a driving mechanism known to those of skill in the art. For example, a triangular, square, or hexagonal drive recess may be incorporated into the head 100 of the bone anchor 10, such that the recess may be engaged by a drive device having a complimentary shape. Other drive recess shapes may correspond to those used with Philips head screwdrivers, slotted screw drivers, star-head drivers, and the like. The bone anchor 10 may also be driven into bone using a driving device having a threaded tip such that the internal threads 130 are engaged by threads on a drive device that then may be removed by reversing the turn of the drive device, thus leaving the bone anchor 10 in the bone.

The bone anchors of the present invention may be made of any material known to those of skill in the art to be compatible with orthopedic usage. For example, the bone anchor may be made of titanium, titanium alloys, stainless steel, stainless steel alloys, chrome cobalt, graphite ceramics, biocompatible polymers, carbon-reinforced composites, and the like. Suitable biocompatible polymers include, but are not limited to, polyethylene (e.g., ultra high molecular weight polyethylene) and polyether ester ketone. The bone anchor may also be coated with materials that promote bone growth such as, but not limited to, hydroxyapatites, plasma spray coatings, porous coatings, and the like. Porous coatings include porous coated alloys such as, but not limited to cobalt chromium alloy or cobalt chromium molybdenum alloy on a porous layer of beads, or porous titanium.

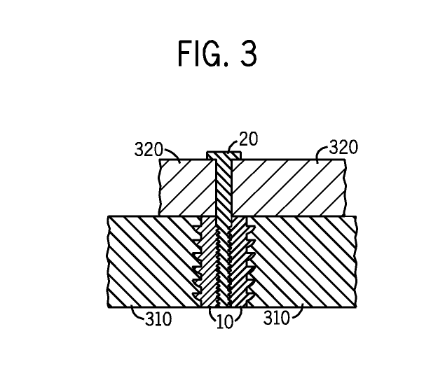

In embodiments where the internal fastener 20 is an internal screw, the threads 240 of the internal fastener 20 are died such that they correspond to the tap of the internal threads 130 of the bone anchor 10. The head 210 of the internal fastener 20 may comprise a driving mechanism known to those of skill in the art. For example, a triangular, square, or hexagonal drive recess may be incorporated into the head 210 of the internal fastener 20, such that the recess may be engaged by a drive device having a complimentary shape. Other drive recess shapes may correspond to those used with Philips head screwdrivers, slotted screw drivers, star-head drivers, and the like. Although the distal end of the internal fastener 20 of FIG. 3 is shown extending all the way to the distal end of the bone anchor 10, it is possible to design the device such that the internal fastener 20 extends only part way down the length of the cannula 110 in the bone anchor 10 when the device is in place in a patient. In these latter embodiments, bone growth into a lower portion of the cannula (below the distal end of the internal fastener) may further assist in fixing the bone anchor to a bone. Bone growth into the lower portion of the cannula may be enhanced by providing fenestrations in the lower portion of the bone anchor to allow bone growth through the fenestrations and into the cannula.

The internal fastener 20 may be made of any material known to those of skill in the art to be compatible with orthopedic usage. For example, the internal fastener 20 may be made of titanium, titanium alloys, stainless steel, stainless steel alloys, ceramics, biocompatible polymers, carbon-reinforced composites, and the like. However, desirably the internal fastener 20 is made of a material that does not allow for bone ingrowth into the material or incorporation of the internal fastener 20 into the bone. In such embodiments, the internal fastener 20 (and any medical device attached thereto) is capable of being removed relatively easily, even if bone ingrowth into the anchor has taken place. An associated medical device and bone anchor device may be referred to as a medical device assembly. In some embodiments, the internal fastener 20 may be a screw similar to those described in U.S. Pat. Nos. 5,474,555 and 5,466,237, however such an internal fastener engages a bone anchor rather than directly engaging bone.

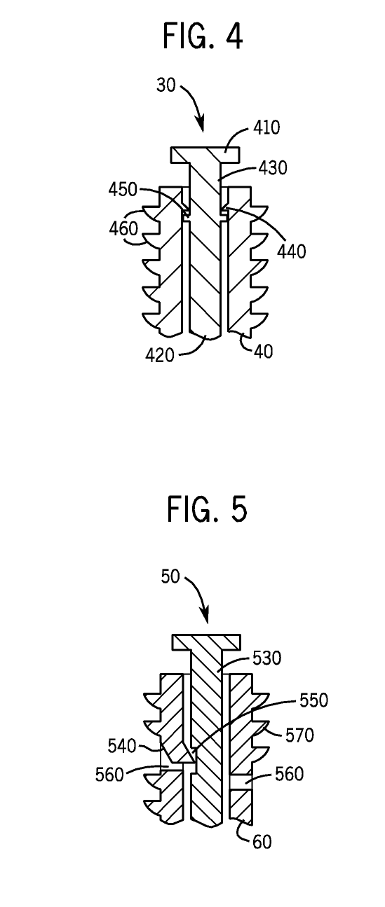

In another aspect, an internal fastener 30 is provided for use with a bone anchor 20, wherein the internal fastener 30 is capable of being permanently engaged in the bone anchor 40 having at least one thread 460 for securing the bone anchor 40 in a bone. One such non-limiting example of a permanently engaging anchoring device is illustrated in FIG. 4, showing the internal fastener 30 and bone anchor 40. A locking mechanism between bone anchor 40 and internal fastener 30 provides the permanent engagement. The internal fastener 30 has a proximal end comprising a head 410 and a distal end comprising a tip 420, the head 410 and the tip 420 being separated by an elongated shaft 430. The external surface of the elongated shaft 430 comprises an annular ring 450. The surface of the cannula of the bone anchor 40 comprises at least one retaining member 440, such that when the internal fastener 30 is inserted in the bone anchor 40 and the annular ring 450 is pushed past the at least one retaining member 440, the internal fastener 30 is prevented from being removed and is permanently engaged in the bone anchor 40. In some embodiments, the annular ring 450 may snap past the retaining member 440, acting as a snap feature. The annular ring 450 provides a continuous surface with which the internal fastener 30 is retained in the bone anchor 40 by the at least one retaining member 440, such that turning of the internal fastener 30 does not result in its removal. In such embodiments, a plurality of annular rings and retaining members may be used. The option of such embodiments, removable and permanent engagement of an internal fastener in a bone anchor, allow one of skill in the art to determine in advance if a removable internal fastener or a permanently engaged internal fastener is required.

FIG. 5 is another, non-limiting illustration of permanently engaging an internal fastener 50 in a bone anchor 60. As shown, the internal fastener 50 has an elongated shaft comprising at least one recess 550, the bone anchor 60 has at least one retaining member 540, and the bone anchor 60 may also comprise at least one fenestration 560 and threading 570 for securing the bone anchor 60 in a bone. In such embodiments, the at least one recess 550 engages the at least one retaining member 540 such that removal of the internal fastener 50 from the bone anchor 60 is prevented. A plurality of recesses and retaining members may be used.

Alternatively, the locking mechanism may be a lock and key type mechanism. For example, the elongated shaft of an internal fastener may have a cross-sectional shape other than completely round and a cannula of a bone anchor has a shape that is complementary, such that when the internal fastener is inserted into the bone anchor, rotation of the internal fastener is prevented by the shape matching. In such cases, permanent attachment of the internal fastener in the bone anchor may be achieved by providing at least one retaining member on the surface of the elongated shaft of the internal fastener and at least one retaining member on the internal surface of the cannula in a position that will engage the at least one retaining member.

Internal fasteners intended for permanent engagement in a bone anchor may or may not be made of materials that allow for bone ingrowth. For example, the internal fastener may be made of the materials identified above for those internal fasteners that do not allow for bone ingrowth, or they may be made of materials similar to the bone anchor in which bone growth is promoted and encouraged by porous surfaces and porous coatings.

In some embodiments, the internal fastener may be used to reduce fractures. For example, a bone anchor may be secured in a bone and an internal fastener inserted through another bone, or fragment of bone, the internal fastener then being engaged within the bone anchor to reduce the fracture. Such internal fasteners may also be an intramedullary (IM) nail. IM nails are typically inserted lengthwise through bone to secure and realign broken or misshapen bones. The internal fastener, or IM nail, may be inserted through a bone lengthwise and inserted into a bone anchor secured in another bone such that when the IM nail extends through the bone, it may be permanently engaged in the bone anchor, thus securing the bone requiring realignment or reshaping. Such an engagement allows a surgeon to make the connection without the need for securement by transverse pins through the bone and into the IM nail.

In another aspect, methods of using the bone screw device are provided. In one method, the bone anchor 10 is driven into a bone 310, as illustrated in FIG. 3 using an appropriate drive device. A medical device 320 is then placed over the head 100 and the cannula 110 of the bone anchor 10 and the internal fastener 20 is inserted through the medical device 320 and into the cannula 130 before being secured in the bone anchor 10 by driving until tight. Over time, a bone reduction will set and heal or a medical device may become unnecessary or need to be replaced or repaired. However, due to the porous coating or materials used to construct the bone anchor 10, removal of that part of the device may be difficult without causing severe trauma to the area. The method thus may further comprise removing the internal fastener 20 from the bone anchor 10, thereby freeing the device 320 from the bone, while leaving behind a minimal amount of foreign material (i.e., bone anchor 10) in the subject. Because the internal fastener 20 does not allow for bone ingrowth, removal of the internal fastener 20 may cause less trauma to the bone and surrounding tissue. Bone may then continue to grow into the cannula 110 of bone anchor 10, thus incorporating the bone 10 anchor wholly into the bone over time. Alternatively, an internal fastener 20 may be reinserted into the bone anchor 10 after a device has been removed, so that the cannula in the bone anchor remains bone-growth-free and may be reused to re-anchor another medical device, should the need arise. In other embodiments, the internal fastener 20 is inserted through a bone prior to insertion into the bone anchor 10 to reduce a fracture.

In another embodied method, the bone anchor 10 is driven into a bone 310, as illustrated in FIG. 3 using an appropriate drive device. The internal fastener 20 is then inserted into the cannula 130 before being secured in the bone anchor 10 by driving until tight with the head 210 of the internal fastener 20 adjacent to the head 100 of the bone anchor 10. The result is a completely nested internal fastener in bone anchor, without an attached medical device.

In yet another embodied method, the bone anchor 40 is driven into a bone, using an appropriate drive device. The internal fastener 30 is then inserted into the cannula of the bone anchor 40, and is pushed in to such a depth that the annular ring 450 passes the at least one retaining member 440. The result is that the internal fastener 30 is permanently engaged within the bone anchor 40. In such embodiments, the internal fastener may be used to reduce a bone fracture, or align a bone. In embodiments where the internal fastener 30 and bone anchor 40 secure a medical device, the internal fastener 30 is first inserted through an attachment bore on the medical device before inserting into the bone anchor 40.

Referring to FIG. 5, in yet another method, the internal fastener 50 is inserted into the bone anchor 60 such that the at least one retaining member 540 engages the at least one recess 550 thereby preventing removal of the internal fastener 50 from the bone anchor 60.

Examples of medical devices that may be anchored to bones with one or more of the present devices include, but are not limited to, spinal stabilization devices, such as those described in U.S. Patent Application Publication No. 2005/0113927, intervertebral disc prostheses, such as those described in U.S. Patent Application Publication No. 2005/0071007, and prosthetic vertebral bodies, such as those described in U.S. Patent Application Publication No. 2005/0137707. For example, a stabilizing rod or plate may be connected to an internal fastener which takes the form of the “anchor,” as described in U.S. Pat. No. 5,474,555 or U.S. Pat. No. 5,466,237, and the internal fastener may then be secured to a bone anchor, rather than directly engaging a bone. In some embodiments, the medical device that may be anchored to a bone is a rod, a plate, a disc, a cervical device, a lumbar device, or other device known to those of skill in the art. The bones with which the bone anchor devices of the present invention may be used, include bones not only associated with the spinal column, but any bones within the body. For example, the bones to be secured or to which a medical device may be secured by bone anchor devices of the present invention include bones associated with, but not limited to, the arms, legs, spinal column, ribs, skull, hip, knee, ankle, foot, and hand.

For the purposes of this disclosure and unless otherwise specified, “a” or “an” means “one or more”. All patents, applications, references and publications cited herein are incorporated by reference in their entirety to the same extent as if they were individually incorporated by reference.

While some detailed embodiments have been illustrated and described, it should be understood that such detailed embodiments are merely exemplary and changes and modifications can be made therein in accordance with ordinary skill in the art without departing from the invention in its broader aspects as defined in the following claims.

1. An anchoring device comprising:
(a) a bone anchor comprising an elongated shaft having a threaded external surface and an internal surface that defines a cannula, the shaft comprising a proximal end and a distal end, wherein at least a portion of the external surface of the bone anchor (i) is made from, or coated with, a porous material that allows for bone ingrowth; (ii) comprises fenestrations, channels or cavities that allow for bone ingrowth; or both (i) and (ii); and
(b) an internal fastener comprising an elongate shank having a proximal end and a distal end;
wherein the internal fastener is capable of being removably engaged in the cannula of the bone anchor.

2. The device of claim 1, wherein
the internal fastener is an internal screw comprising a threaded external surface on the elongated shaft;
the internal surface of the bone anchor comprising a threaded surface; and
the threaded external surface of the internal screw matches the threaded internal surface of the bone anchor.

3. The device of claim 1, wherein the internal fastener is made from a non-porous material that does not allow for bone ingrowth.

4. The device of claim 1, wherein the bone anchor further comprises at least one fenestration traverse to the cannula.

5. The device of claim 1, wherein the bone anchor has a porous coating on at least a portion of its external surface.

6. The device of claim 5, wherein the porous coating comprises a material selected from the group of materials consisting of cobalt chromium alloy, cobalt chromium molybdenum alloy, or porous titanium.

7. The device of claim 1, wherein the cannula extends through the elongated shaft from its proximal end to its distal end.

8. The device of claim 7, wherein the distal end of the internal fastener does not extend to the end of the cannula when the device is in place in a patient.

9. The device of claim 8, wherein the elongated shaft of the bone anchor defines one or more fenestrations in a portion of the shaft below the distal end of the internal fastener.

10. A medical device assembly comprising, a medical device secured to at least one anchoring device according to claim 1.

11. The medical device assembly of claim 10, wherein the medical device comprises a spinal stabilization device comprising at least one rod or plate, and further wherein the at least one rod or plate is secured to the internal fastener of the anchoring device.

12. The medical device assembly of claim 10, wherein the medical device comprises a prosthetic intervertebral disc adapted to be fixed between two vertebra.

13. The medical device assembly of claim 10, wherein the medical device comprises a prosthetic vertebral body adapted to be fixed between two vertebra.

14. A method of inserting a bone fastener into a bone using the anchoring device of claim 1, the method comprising inserting the bone anchor in a bone and inserting the internal fastener into the cannula of the bone anchor.

15. The method of claim 14, wherein
the internal fastener is an internal screw, the elongate shaft comprising a threaded external surface;
the internal surface of the bone anchor comprising a threaded surface;
the threaded external surface of the internal screw matches the threaded internal surface of the bone anchor; and
the inserting the internal fastener into the cannula comprises screwing the internal screw into the cannula.

16. The method of claim 14, further comprising fixing a medical device to the bone using the internal fastener.

17. The method of claim 16, further comprising removing the internal fastener from the anchor, thereby releasing the medical device from the bone.

18. The method of claim 16, wherein the medical device is a spinal stabilization device comprising at least one rod or plate and the bone is a vertebra, and further wherein the internal fastener is secured to the at least one rod or plate.

19. The method of claim 16, wherein the medical device comprises at least one of a prosthetic intervertebral disc or a prosthetic vertebral body and the bone comprises a vertebra.

20. The method of claim 16, wherein the medical device comprises at least one of a rod or a plate.
